(19) United States
(12) Patent Application Publication
(10) Pub. No.: US 2019/0314626 A1
(43) Pub. Date: Oct. 17, 2019

(54) APPARATUS FOR OUTPUTTING ELECTRIC STIMULATION PULSES

(71) Applicant: WAT MEDICAL TECHNOLOGY INC., Ningbo (CN)

(72) Inventors: Meng ZHANG, NINGBO (CN); JUN ZHANG, NINGBO (CN); HAOJIE XU, NINGBO (CN); WUFENG JI, NINGBO (CN); JUN Cao, NINGBO (CN)

(73) Assignee: WAT MEDICAL TECHNOLOGY INC.

(21) Appl. No.: 15/992,179

(22) Filed: May 30, 2018

(30) Foreign Application Priority Data
Apr. 16, 2018 (CN) 201810337724.3

(51) Int. Cl. A61N 1/04; A61N 1/36

(52) U.S. Cl. CPC A61N 1/0456; A61N 1/0472; A61N 1/36034

(57) ABSTRACT

The present disclosure discloses an apparatus for outputting an electric stimulation pulse comprising an outer housing, an inner housing, two extending parts, two electrodes, a switch and a controller. The switch outputs a first input pulse and a second input pulse separated apart from the first input pulse by a time interval to the controller. The controller is configured to output an output pulse to the electrodes upon receiving the first input pulse which rises in intensity over a first period of time. The first period of time is the smaller of the time interval and a predetermined time threshold. Embodiments of the present disclosure provide an easy-to-use device for outputting electrical stimulation pulses that can reduce the user's discomfort due to current stimulation when mitigating migraine.

### CROSS-REFERENCE TO RELATED APPLICATIONS

This application claims priority to Chinese Patent Application No. 201810337724.3 with a filing date of Apr. 16, 2018. The content of the aforementioned applications, including any intervening amendments thereto, are incorporated herein by reference.

### TECHNICAL FIELD

The present disclosure relates to the field of wearable medical devices, and more particularly, to an apparatus for outputting electric stimulation pulses.

### BACKGROUND OF THE PRESENT INVENTION

Transcutaneous electrical nerve stimulation (TENS) is a non-invasive and non-invasive analgesic method for blocking pain sensory nerve signals by stimulating nerves with electric pulses, and is usually used to relieve various types of pain sensations such as dysmenorrhea, migraine, and vertigo. The device for performing the TENS therapy would contact the user's skin and output electric pulses that vary according to the type of pain suffered by the user to introduce therapeutic effects. Various parameters such as the pulse duration, frequency, intensity, period, and intensity change slope of the output electrical pulse need to be configured in an optimized range so as to provide preferable analgesic effects and reduce the user's discomfort. Most of head pains, such as migraine, are transmitted on the supraorbital nerve and the supratrochlear nerve. Delivering an appropriate electrical signal at locations corresponding to these nerves of a patient would likely relieve the headache signals transmitted on these nerves without requiring the patient to take medication.

### SUMMARY OF PRESENT INVENTION

An objective of the present disclosure is to provide a more convenient device for outputting an electrical stimulation pulse that can reduce the user's discomfort while mitigating headaches.

The present disclosure discloses an apparatus to output an electric stimulation pulse, including an outer housing having two symmetrically distributed extending parts, an inner housing enclosed by the outer housing, a controller mounted within the inner housing, each extending part having an electrode electrically coupled to the controller, and a switch electrically coupled to the controller to output a first input pulse and a second input pulse separated apart from the first input pulse by a time interval to the controller.

The controller is configured to output an output pulse to the electrodes upon receiving the first input pulse. The output pulse rises in intensity over a first period of time. The first period of time is the smaller of the time interval and a predetermined time threshold.

In some embodiments, the intensity of the output pulse during a second period of time following the first period of time equals to the intensity of the output pulse at the end of the first period of time.

In some embodiments, the pulse duration of the second input pulse is less than the pulse duration of the first input pulse.

In some embodiments, the second period of time ends upon receiving a third input pulse, the pulse duration of the third input pulse being greater than the pulse duration of the second input pulse.

In some embodiments, the pulse duration of each of the first input pulse and the third input pulse is greater than twice the pulse duration of the second input pulse.

In some embodiments, the apparatus further comprises a buzzer for outputting sound signals with durations proportional to the pulse durations of the first input pulse, the second input pulse and the third input pulse respectively when the first input pulse, the second input pulse and the third input pulse are received.

In some embodiments, the apparatus further comprises a light emitting unit for emitting light upon receiving the first input pulse and stopping light emission upon receiving the third input pulse.

In some embodiments, the pulse duration of the third input pulse is greater than the pulse duration of the first input pulse.

In some embodiments, the switch is a push switch, and the pulse duration of each of the first input pulse, the second input pulse, and the third input pulse is equal to the time length the push switch is held pressed, respectively.

In some embodiments, the output pulse has a frequency of 60 Hz, a pulse duration of 250 microseconds, and a maximum intensity of 16 milliamps.

In some embodiments, the sum of the first period of time and the second period of time is 20 minutes.

In some embodiments, the predetermined time threshold is 5 minutes.

The present disclosure discloses a method to output electric stimulation pulses, including providing an electric stimulation apparatus comprising a controller, a switch electrically coupled to the controller, and two electrodes electrically coupled to the controller, receiving, by the controller, a first input pulse and a second input pulse separated apart from the first input pulse by a time interval, and outputting an output pulse by the controller to the electrodes upon receiving the first input pulse. The output pulse rises in intensity over a first period of time. The first period of time is the smaller of the time interval and a predetermined time threshold.

The embodiments of the present disclosure allow the user to control the electrical stimulation pulses used when performing electrical stimulation in a simpler and more comfortable manner, and the user can also perceive the TENS treatment process through optically and acoustically intuitive feedback so that he or she could find the most proper control parameters for his or her personal TENS treatment session.

### DESCRIPTION OF THE DRAWINGS

The present disclosure is illustrated by way of example and not limitation in the figures of the accompanying drawings, in which like references indicate similar elements, and in which:

FIG. 1 is a top view of an apparatus to output an electric stimulation pulse according to an embodiment.

FIG. 2 is a bottom view of an apparatus to output an electric stimulation pulse according to an embodiment.

FIG. 3 is an explosive view of an apparatus to output an electric stimulation pulse according to an embodiment.

FIG. 4 is a block diagram of an apparatus to output an electric stimulation pulse according to an embodiment.

### DETAILED DESCRIPTION OF PREFERRED EMBODIMENTS

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components. but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element. component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

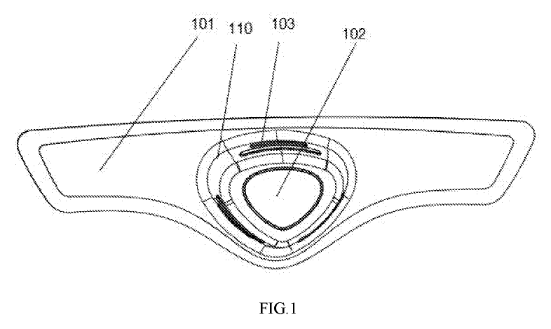

FIG. 1 is a top view of an apparatus to output an electric stimulation pulse according to an embodiment. The outer housing 110 of the apparatus has two substantially symmetrically distributed extending parts 101. The two extending parts 101 may be fixed on both sides of the outer housing 110. The two extensions 101 are made of flexible materials such as silicone, so that they can be bent to fit the shapes of foreheads of different users. The outer housing 110 has a switch 102. The switch 102 can be, for example, a push switch for generating an electrical pulse when pressed by a user. The time length or pulse duration of the electrical pulse is equal to the time the user hold the switch 102 depressed. One or more protrusions 103 are provided on the outer housing 110 to facilitate the user to grasp the outer housing 110. The protrusions 103 may be arranged around the switch 102.

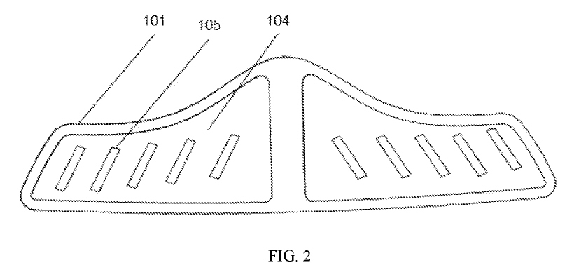

FIG. 2 is a bottom view of an apparatus to output an electric stimulation pulse according to an embodiment. One electrode 104 is arranged on the back of each extending part 101. The two electrodes 104 would be in contact with the user's skin and apply pulses for electrical stimulation to the user. As shown in FIG. 2, each electrode 104 has a plurality of elongated notches 105. The size of each notch 105 is uniform and the distance between each pair of adjacent notches 105 is also uniform. The notches 105 are configured to allow the current density to be more evenly distributed on the electrodes 104 and also enable a better ohmic contact between the electrodes 104 and the user's skin. Although the shape of the notch 105 in the figures is shown as an approximately linear shape, those skilled in the art will understand that the notch 105 may be provided in other shapes such as an arc shape.

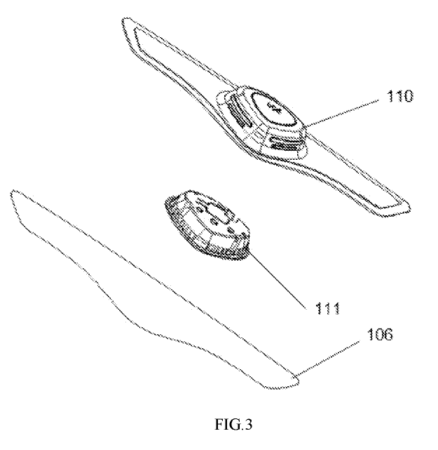

FIG. 3 is an explosive view of an apparatus to output an electric stimulation pulse according to an embodiment. An inner housing 111 is arranged within the outer housing 110. The inner housing 111 may be enclosed in a package formed by the outer housing 110 and the base plate 106. The electrode 104 is electrically coupled to the circuit boards, the power supply, and other components contained in the inner housing 111 through the base plate 106. The base plate 106 may be a conductive pad with adhesive materials such that it can used to conduct and attach the apparatus to the head. The bottom plate 106 can be separated from the apparatus or integrated into the apparatus. A portion of the inner housing 111 can be pressed down under external force, and reset to its original position when the external force is lifted, so that it can become in contact with the switch 102. The inner housing 111 can provide additional protection and insulation for any internal circuit board and power supply. In order to reduce the size of the apparatus and improve safety, it is preferable that no interface for performing operations such as charging be provided on the outer housing 110. In addition to the electrode 104, the apparatus that outputs the electrical stimulation pulse includes no other contacts that can bring an electrical contact. Both the inner housing 111 and the outer housing 110 may be constructed in a non-detachable structure and sealed from outer environment. The power source in the inner housing 111 can be implemented using various types of batteries. The service life of the power source can be extended by controlling the intensity of the output pulse without having to manually recharge it again.

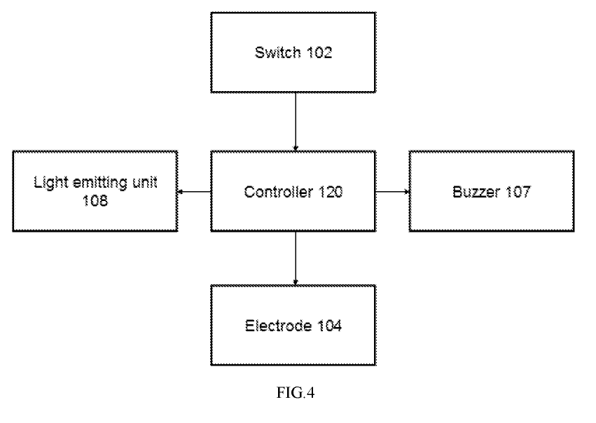

FIG. 4 is a block diagram of an apparatus to output an electric stimulation pulse according to an embodiment. A controller 120 is disposed in the inner housing 111, which can be any specific or general-purpose processor, controller chip, SCM, or the like. Optionally, sensors such as resistance sensors and temperature sensors may also be arranged in the inner housing 111 to sense the conditions of the user and the environment. The controller 120 can be electrically coupled to the electrodes 104 to output electric stimulation pulses. Well-known inverters, transformers, and other components not shown in the figure may be arranged between the controller 120 and the electrodes 104 to adjust the waveform of the output pulses. The controller 120 is also electrically coupled to the switch 102 to receive user input. When using the device for outputting electrical stimulation pulses, the user outputs two input pulses temporally separated from each other to the controller 120 by operating the switch 102. The first input pulse of these two input pulses is used to instruct the controller 120 to start outputting the output pulses to the electrodes 104. The output pulse can have a two-phase rectangular waveform or a spike-like waveform, which has a frequency of 50-200 Hz, and for example, 60 Hz. The pulse duration can be 100-500 microseconds, and for example. 250 microseconds. After receiving the first input pulse, the controller 120 will linearly increase the intensity of the output pulse over time. The intensity of the output pulse will reach a maximum value after a predetermined time threshold, and thereafter remain at that maximum value. The maximum value is, for example, 8-16 mA, and the time threshold is, for example, 3-14 minutes. In one embodiment, the maximum value of the intensity of the output pulse is 16 mA and the predetermined time threshold is 5 minutes. At this time, the intensity of the output pulse can gradually increase with a slope of 53.3 μA/sec. Such a slowly rising slope allows the nervous system to gradually raise the threshold of pain perception and avoids a sudden experience of pain perceived by the user. Excessive rising slopes may cause tingling to the user, while slopes that are too low may waste more of the user's time during waiting.

If a second input pulse which has a time interval less than the time threshold from the first input pulse is received after receiving the first input pulse, the intensity of the output pulse upon receiving the second input pulse would not have reached the maximum value allowed yet. The increase in the output pulse intensity would be stopped in this situation. When the intensity of the output pulse stops increasing due to receiving the second output input pulse, the controller 120 will output an output pulse thereafter which has an intensity equal to the intensity at the time of receiving the second input pulse. The user can control the intensity of the output pulse to be finally reached by adjusting the time interval between the two input pulses.

The user may send a third input pulse through the switch 102 again after the second input pulse to instruct the controller 120 to stop outputting the electrical stimulation pulse. Even if the third input pulse is never to be received, the controller 120 will automatically stop outputting the output pulse after a certain period of time. For example, the controller 120 will stop outputting the output pulse 20 minutes after the first output pulse is received, regardless of whether or not another input from the user would be received. If the user needs to manually shut down the apparatus, a third input pulse can be manually input and the session can be stopped before reaching the 20-minutes limit. In order to distinguish different types of user input, the pulse duration of the second input pulse for setting the pulse intensity is set to be less than the pulse duration of the first input pulse, and the pulse duration of the third input pulse can be greater than the pulse duration of the second pulse. The user needs to press the switch 102 for more than 2 seconds to turn the device on or off, while a pressing time of less than 1 second can be defined for determining the final pulse intensity. The time used for pressing the switch 102 will be equal to the pulse duration of an input pulse generated by the switch 102. In order to make a substantial distinction, the pulse duration of each of the first input pulse and the third input pulse may be greater than twice the pulse duration of the second input pulse. The pulse duration of the third input pulse may be greater than the pulse duration of the first input pulse, such that the controller 120 may determine to shut down the apparatus when receiving another input pulse having a pulse duration greater than the first input pulse after receiving the first input pulse.

The controller 120 is also optionally connected to the buzzer 107 for outputting a sound signal with a time duration proportional to the pulse duration of each of the first input pulse, the second input pulse and the third input pulse, respectively when the first input pulse, the second input pulse and the third input pulse are received, so that the user may be reminded of operations he or she has performed. The buzzer 107 can sound in different sound patterns, such as different numbers of beeps, when receiving the first input pulse, the second input pulse and the third input pulse. The controller 120 is also optionally connected to a light emitting device 108 such as an LED. In some embodiments, the light emitting device 108 starts emitting light upon receiving the first input pulse and stops emitting light upon receiving the third input pulse in order to indicate the current operating state. In some embodiments, the light emitting device 108 may include a combination of LED lights of various colors and emit light of different colors according to the remaining power of the power source. In some embodiments, the light emitting device 108 may blink at a higher frequency when the first input pulse and the third output pulse are received, and blink at a lower frequency when the second input pulse is received.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

We claim:

1. An apparatus for outputting an electric stimulation pulse, including:
an outer housing having two symmetrically distributed extending parts;
an inner housing enclosed by the outer housing;
a controller mounted within the inner housing, each extending part having an electrode electrically coupled to the controller; and
a switch electrically coupled to the controller to output a first input pulse and a second input pulse separated apart from the first input pulse by a time interval to the controller, wherein
the controller is configured to output an output pulse to the electrodes upon receiving the first input pulse, the output pulse rising in intensity over a first period of time, the first period of time being the smaller of the time interval and a predetermined time threshold.

2. The apparatus of claim 1, wherein the intensity of the output pulse during a second period of time following the first period of time equals to the intensity of the output pulse at the end of the first period of time.

3. The apparatus of claim 2, wherein the pulse duration of the second input pulse is less than the pulse duration of the first input pulse.

4. The apparatus of claim 3, wherein the second period of time ends upon receiving a third input pulse, the pulse duration of the third input pulse being greater than the pulse duration of the second input pulse.

5. The apparatus of claim 4, wherein the pulse duration of each of the first input pulse and the third input pulse is greater than twice the pulse duration of the second input pulse.

6. The apparatus of claim 5, further comprising a buzzer for outputting sound signals with durations proportional to the pulse durations of the first input pulse, the second input pulse and the third input pulse respectively when the first input pulse, the second input pulse and the third input pulse are received.

7. The apparatus of claim 6, further comprising a light emitting unit for emitting light upon receiving the first input pulse and stopping light emission upon receiving the third input pulse.

8. The apparatus of claim 7, wherein the pulse duration of the third input pulse is greater than the pulse duration of the first input pulse.

9. The apparatus of claim 8, wherein the switch is a push switch, and the pulse duration of each of the first input pulse, the second input pulse, and the third input pulse is equal to the time length the push switch is held pressed, respectively.

10. The apparatus of claim 9, wherein the output pulse has a frequency of 60 Hz, a pulse duration of 250 microseconds, and a maximum intensity of 16 milliamps.
